(19) United States
(12) Patent Application Publication
(10) Pub. No.: US 2018/0369015 A1
(43) Pub. Date: Dec. 27, 2018

(54) THERMAL THERAPY DEVICE FOR PROVIDING CONTROLLED HEATING AND COOLING VIA AN APPLIED TISSUE INTERACTING DEVICE

(71) Applicant: Appliance Development Corporation, Danvers, MA (US)

(72) Inventors: Dov Z. Glucksman, Winchester, MA (US); Andrew Joseph Rojee, Derry, NH (US); William John Rittman, III, Wellington, FL (US); Marsha Lee Calise, Wellington, FL (US)

(21) Appl. No.: 16/014,147

(22) Filed: Jun. 21, 2018

Related U.S. Application Data
(60) Provisional application No. 62/523,821, filed on Jun. 23, 2017.

(51) Int. Cl. A61F 7/00

(52) U.S. Cl. CPC A61F 7/0085; A61F 7/007; A61F 2007/0225; A61F 2007/0054; A61F 2007/0071

(57) ABSTRACT

A thermal therapy device has a fluid manipulating device for thermally manipulating and circulating therapy providing fluid via flexible conduit to a tissue interacting device. The thermal therapy device utilizes a thermoelectric cooling device including a chilled fluid reservoir for extracting heat from the tissue being treated during the cold therapy cycle and utilizes a resistive electric heater for heating the tissue being treated during the hot therapy cycle.

### RELATED APPLICATION

This Application claims all benefit and priority to U.S. Provisional Application No. 62/523821 filed on 23 Jun. 2017, the entire teachings of which are incorporated herein by reference.

### FIELD OF THE INVENTION

The present invention generally relates to an electrical thermal therapy device having a tissue interacting device applied to the patient, and a fluid manipulating device for circulating therapy providing fluid through the tissue interacting device.

### BACKGROUND

The topical application of heating or cooling to human tissue using pads or blankets for the treatment of injuries and pain have been used for a long time. Hot/cold therapy is also known to be used to improve the flexibility of tendons and ligaments, reduce muscle spasms and alleviate pain.

Heat therapy is the heating of tissue by using various techniques, such as hot water bottles or cloth soaked in hot water, blankets or pads heated by internal electrical resistive heating, or the application of ultrasound energy. Heat therapy leads to vasodilation, which in turn increases the blood flow in the affected tissues. The increased blood flow in the target area carries with it extra oxygen and other nutrients, thus accelerating the healing process. Additionally, the application of heat reduces muscle spasm and relaxes stretched muscles leading to pain relief. Heat therapy is generally used to treat chronic pain such as low back pain, spinal, neck pain, neuropathic pain, and other muscular spasms. Heat therapy is generally applied at a temperature range of 40-50° C.

Cold therapy was historically accomplished by using ice or a chemical gel. Cold therapy is typically used during the first 24 to 48 hours following an injury, typically to get relief from bruises, bumps and sprains. Cold therapy calms down damaged tissue causes vasoconstriction, which reduces blood circulation and numbs the nerves, decreasing inflammation, pain, and muscle spasm. Cold therapy is generally used to treat acute pain caused due to injuries such as runner's knee and freshly pulled muscle. Cold therapy is generally applied at a temperature range of 5-10° C.

Both therapies have the advantage of being effective for the treatment of swelling and pain while being non-addictive and non-invasive.

There is also growing use of contrast therapy. It is performed through the intermittent application of hot and cold packs on the skin of an injured area. It decreases pain, increases circulation, and speeds healing. Contrast therapy is most often used to treat sports injuries, but it is also used on chronic or repetitive injuries and injuries in the stages of healing. Contrast therapy generally consists of applying ice, then heat, then ice again in a repetitive manner. Typical ratios call for applying heat for twice as long as applying cold. One always wants to end the process with a cooling application. Typical treatment time is 30 minutes.

The global hot and cold therapy market is anticipated to be driven by factors such as steep growth in chronic musculoskeletal disorders, increase in global trauma and accident cases, and rise in inclination for non-invasive pain management approaches. Increase in the geriatric population vulnerable to chronic pain is also escalating demand for hot and cold therapy treatment, globally.

Increasing incidence of lifestyle related disorders is one of the major drivers of the use of hot and cold therapy. This is significantly driven by the growing awareness about the long-term side effects caused using prescription and over-the-counter drugs. In terms of available products, the hot and cold therapy market can be divided into dry and moist hot and cold packs or compresses, gel packs, and electric hot/cold pads. These are used to manage various conditions including back/spinal problems, neck problems, joint problems, neuropathic pain, cold tumors, poliomyelitis, pelvic diseases, malignant ulcers, hemostasis, analgesic, soft tissue inflammation, sports injuries and post-surgery pain.

There are many drawbacks to the products currently on the market that compromise their application. Regarding heating, there are several techniques used to create a hot applicator. For instance, some packs are designed to be microwaved, which suffer from drawbacks such as difficulty in controlling the temperature, which can become too hot causing burns/cellular damage; they also lose temp rapidly, necessitating their ongoing reheating. Chemical packs are also commonly used, but they also have limitations based on lack of temperature control; they can leak and are therefore prone to cause chemical burns; they tend to be expensive for long term use, being disposable. Probably the most effective and safe heating technique up until now was the use of an electric heating pad.

Regarding cooling, ice packs that are kept in the freezer are most commonly used. Their shortcomings are due to difficulty to control temperature—the affected area can become too cold causing possible cold burns/cellular damage, they heat up rapidly, requiring to be frequently exchanged with a freshly cooled pack and placed back in the freezer to be refrozen. Chemical ice packs have the same drawbacks as the chemical heating packs. Actively Pumped chilled water units for cold therapy are bulky, require ice and water on hand. Further, the water can spill/leak, and there is no true temperature control.

To offer a combined heating and cooling therapy (contrast therapy) using these standard products would obviously require the purchase of two separate sets of products thus being expensive, requiring extra storage space and consuming a lot of time during application. To overcome these issues some devices are available that combine these two therapies into one product.

As an example, U.S. Pat. No. 9,283,109 assigned to Innovative Medical Equipment, LLC discloses a thermal therapy device, having a tissue-interacting device designed to target one or more significant areas of the therapy-receiving person's body by concentrating the circulation of the therapy providing fluid in these areas and a fluid-manipulating device having a thermoelectric module coupled to a heat exchanger a pump, a fan and a housing enclosing these components. The heat exchanger heats/cools the therapy providing fluid, and the pump circulates the therapy providing fluid through the system. To facilitate the heating and cooling of the therapy providing fluid the control circuit is designed to reverse the polarity of the current applied to the thermoelectric element, thereby with one polarity heat is extracted from the fluid whereas with the opposite polarity heat is delivered to the fluid.

It was observed through tests conducted with Innovative Medical Equipment, LLC Model 18506-KK (being the only product found on the market claiming to be exercising the '109 Patent disclosed structure) that it takes a long time for the tissue interacting device to cool down after the polarity is switched from the heating to the cooling portion of the therapy, and that it also takes a long time for the tissue interacting device to heat up after the polarity is switched from the cooling to the heating portion of the therapy.

Therefore, a need exists for a device and a method for rapidly heating and cooling tissue interacting device, where the apparatus is compact and affordable. The apparatus and method presented in this invention incorporates a resistance heater for heating and a thermoelectric element for cooling as well as storage compartment for cooled liquid as well as other unique features as will be described here.

### SUMMARY OF THE INVENTION

Described herein is a novel electric thermal therapy device that combines tissue interacting device and a fluid manipulating device, being interconnected via a flexible conduit for circulating therapy providing fluid thereby enabling the tissue interacting device to supply to or to extract heat from tissue to which it is applied for treatment. The fluid manipulating device, has a thermoelectric cooling unit which extracts heat from the therapy providing fluid as it is pumped through the fluid manipulating device, using an electric pump also enclosed in the fluid manipulating device. The cold side of the thermoelectric cooling unit and the pump are encased in insulation foam to prevent heat being absorbed from the environment. The fluid manipulating device, also has control circuit including temperature and time controls, accessible to the user or operator for adjusting temperatures and treatment times of the tissue interacting device, and for selection of thermal therapy mode, being: heating therapy, cooling therapy or contrast therapy. The tissue interacting device may have a flexible treatment element having a meandering conduit, containing therapy providing fluid configured with at least one continuous flow channel, tissue interacting device is made to conform to the shape of the body part being treated to effectively transfer heat to and from it; the tissue interacting device may also have a flexible electric heating element; the tissue interacting device may also haves an insulation layer attached to the back of the tissue interacting device to ensure that no heat is lost to or absorbed from the environment. The tissue interacting device may also incorporate a temperature sensor for sensing the temperature of the treatment area, thereby to facilitate the thermal response of the control circuit. The interconnecting flexible conduit may have an insulated outer tube firmly connected at each of its ends to the fluid manipulating device and to the tissue interacting device; and may contain two flexible tubes for circulating the tissue treatment fluid between the fluid manipulating device to the tissue interacting device, it may also contain two electric conductors to provide electric power to the heating element and may also contain two leads for connecting thermal sensor to the control circuit for transmitting information pertaining to the the temperature of the treatment area.

It was determined that by using a separate means to cool and to heat the treatment area, the delays between cooling and heating cycles may be greatly reduced, enhancing the user's satisfaction with the device.

The invention may be embodied in or practiced using a thermal therapy device having a tissue interaction portion and a fluid manipulation portion in fluid communication therewith by conduit; wherein the tissue interacting portion, fluid manipulating portion, and conduit form a closed loop containing a fluid and are adapted to cause the fluid to flow through the closed loop. The fluid may be heated by a heating source and cooled by a cooling source, the heating and cooling sources being distinct from each other, and the tissue interaction portion may be adapted to heat and cool patient tissue and may include a temperature sensing device to monitor the temperature of a treatment area and control the heating and cooling sources according thereto.

The fluid manipulating portion may include the heating source, the cooling source, and a pump for causing the fluid to flow through the closed loop. Alternatively, the fluid manipulating portion may include the heating source and a pump for causing the fluid to flow through the closed loop, and the tissue interaction portion may include the heating source.

The closed loop may include channels disposed within the tissue interaction portion and adapted for thermal communication between the fluid and the patient tissue. The heating source may be a resistive electric heater. The cooling source may be a thermo electric cooler. The tissue interactive portion include a flexible pad.

Further features and aspects of the invention are disclosed with more specificity in the detailed description and drawings provided herein.

### BRIEF DESCRIPTION OF THE DRAWINGS

FIG. 1 is an isometric view of the thermal therapy device according to the invention;

FIG. 2 is a cross sectional view of a preferred embodiment of a fluid manipulating device according to the invention;

FIG. 3 is an exploded view of the heat exchanger and storage block of FIG. 2 used for extracting heat from therapy providing fluid;

FIG. 4 is a schematic illustration of the thermal therapy device according to the invention; and

FIG. 5 is an exploded view of the tissue interacting device according to the invention.

### DETAILED DESCRIPTION OF AN EXEMPLARY EMBODIMENT

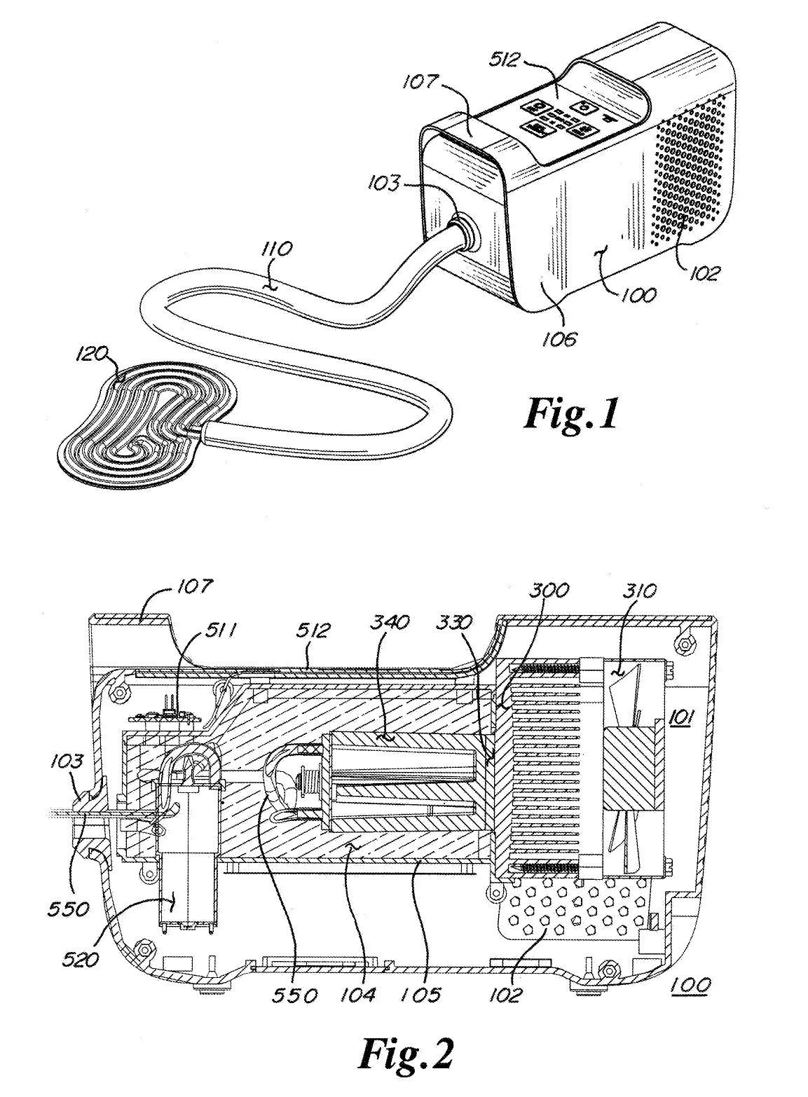

Referring now to FIG. 1, a thermal therapy device is shown. The thermal therapy device includes a fluid manipulating device 100, interconnected via a thermally insulated flexible conduit 110 with a tissue interacting device 120. The flexible conduit 110 is firmly secured to fluid manipulating device via a collar 103. The tissue interacting device can be attached to the treatment area by Velcro strips or other fasteners, not shown. Fluid manipulating device enclosure 106 includes an integral carrying handle 107. Also shown are air outlet openings 102. Fluid manipulating device contains various electrical components used for heating or cooling and circulating the therapy providing fluid; also contained are various electrical and electronic component for operating and controlling the process of heating, cooling and fluid circulation. The tissue interacting device 120 may be placed in direct contact with the appropriate areas of the therapy-receiving person's body so that the device can cool or heat the tissue, as required.

FIG. 2 is a cross sectional view of the fluid manipulation device 100, according to a preferred embodiment of the invention, including an external enclosure 106 containing all the fluid manipulating components, such as the fluid cooling block assembly 340, the thermoelectric element 330, the heat-sink 300 and the cooling fan 310, as well as air inlet 101, and air outlet 102. Also shown are the fluid pump 520, electronic controls 511, and touch key pad 512. Also shown are portions of a fluid conducting flexible tube 550 and flange 103 for connecting to flexible conduit 110. Also illustrated in oblique broken lines is the thermal insulation body 104, which surrounds the cooling components to retard heat transfer from the environment. Thermal insulating body 104 is confined to internal enclosure 105.

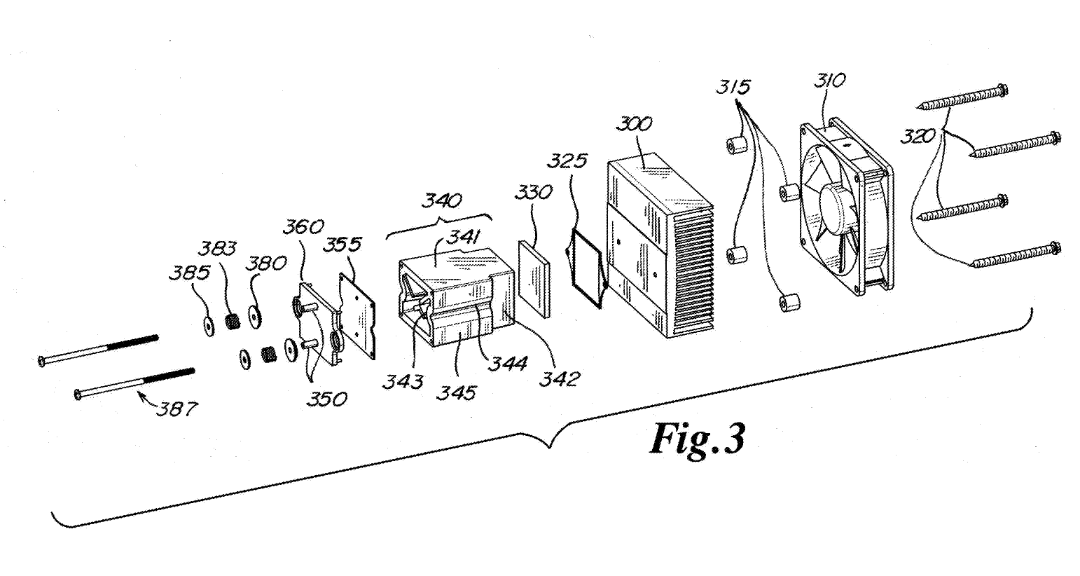

FIG. 3 is an exploded view of the heat exchanger and cooling block assembly 340, intimately connected to the cool face of a thermoelectric cooling device 330, which is secured on all four sides by bracket 325. The hot face of the thermoelectric cooling device 330 is held in intimate contact with the heat sink 300. Heat being transferred from the cooling block plus heat generated by internal losses within the thermoelectric cooling device is conducted away from the hot surface of the thermoelectric cooling device through the heat sink 300, being cooled by air generated by fan 310. The fan is kept at a predetermined distance from the heat sink fins by mounting spacers 315. The fan/heat sink assembly is held together by four self-threading screws 320.

The cooling block assembly 340 includes a cooling block 341 and a cooling block lid 360 sealably held together to form a water tight container. Cooling block main section 345, configured to serve as a fluid storage volume and a smaller cross-sectional portion 342 configured to be of the same size as a thermoelectric cooling element 330 to which is it firmly attached to facilitate optimal heat transfer from the block to the thermoelectric cooling element. The cooling block 341 may be formed by a casting process to create a cavity in its main section 345 with a series of upstanding ribs 343, to increase the heat transfer area between the block 340 and the therapy providing fluid being circulated through the block for cooling. Also shown are two elongated recesses 344 in the main section of the block; they offer space for the securing bolts 387 which provide the tight connection between the cooling block assembly and the heat sink 300, thereby creating a good conductive pass from the cooling block to the thermoelectric cooling element 330 and from the thermoelectric cooling element to the heat sink 300. Thermally conductive paste may be used to provide maximal thermal contact. The cooling block assembly 340 is connected to a fluid circulation path. The cooling block main body 341 is capped by plate 360, with in-between gaskets 355 providing a fluid tight compartment. The circulation path of the cooling fluid also includes the ports 350, extending outwards from the plate 360. The cooling block assembly 340 is attached to the heat sink 300 using two bolts 387 passing through two washers 385, two springs 383, and two insulating bushings 380. The purpose of the springs is to maintain the entire heat transfer system in compression, and to ensure good thermal conductivity regardless of expansion or compression caused by changes in temperature of the components.

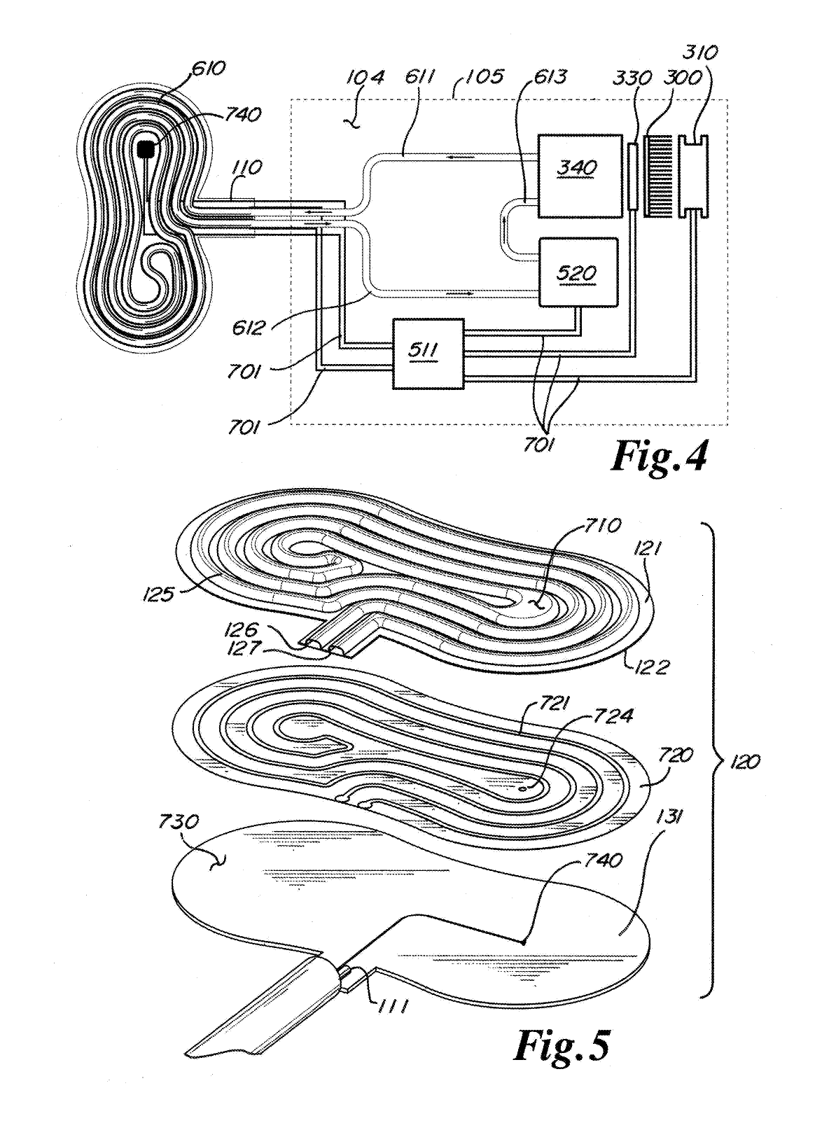

FIG. 4 is a schematic illustration of the thermal therapy device of the first preferred embodiment where tissue interacting device 120 is shown interconnected to the fluid manipulating device 140 through flexible conduit 110 for circulating therapy providing fluid from one to the other for providing treatment to the patient. The schematic illustration shows the operating components described above such as the cooling block assembly 340, the thermoelectric cooling unit 330, the heat-sink 300, the fan 310, the pump 520 and the controls 511. In addition, there is a schematic illustration of the fluid circulation system as well as the electrical wiring.

Also shown are circulation tube 611 through which therapy providing fluid flows from the cooling block 340 to the tissue interacting device 120; from where the therapy providing fluid flows through tube 612 back to the pump 520, and back into the fluid block 340 via tube 613. Solid arrows show the flow direction of the therapy providing fluid within the fluid circulation system. Electronic controls 511 are shown connected via electrical lines 701 to all the electrical components.

FIG. 5 is an exploded view of the tissue interacting device according a preferred embodiment, it shows the fluid circulation portion 710 of the device having two layers of vinyl 121 and 122 or other such flexible plastic sheets fused together in a manner known in the art to form a meandering fluid passage 125, with two openings 126 and 127 for connecting to fluid tubes 111 (only one shown) projecting from flexible conduit 110. Also shown is a flexible heater 720 known in the art as etched or printed heater having a layer of high temperature plastic sheet such as polyimide with a trace of conductive material, such as copper 721, used as a resistive heating element; the ends 722 and 723 of the resistive heater are terminated with electrical conductors, connecting via the flexible conduit 110 to the electrical controls in the fluid manipulating unit.

Also shown is a backing pad 730 of thermally insulating material for preventing any passage of heat through the back of the pad. Additionally, shown is a thermal sensor 740 connected to two leads 701 that project from the flexible conduit 110. Flexible heater 720 includes a cut out 724 for the thermal sensor to get within proximity of the tissue being treated for accurate sensing of the temperature experienced at the treatment area.

As it has become apparent from the description provided above the thermal therapy device according to the present invention employs a thermoelectric cooling device to provide cold therapy and employs electric resistive heaters of one form or another to provide hot therapy. That principle is where the present invention departs from the prior art as exemplified in U.S. '109 patent referenced, which discloses a thermoelectric module being used to extract heat from therapy providing fluid when current flows in one direction through the thermoelectric module, and to heat the therapy providing fluid when current flows in the opposite direction through the thermoelectric module.

The reason for this was based on tests conducted with a thermoelectric module used for both heating and cooling where tests indicated that it took a long time for the temperature of the therapy providing fluid to cool down after being heated. The reason being that the cooling capacity of the average thermoelectric cooling unit is in the range of 40 watt, and that the combined mass of the elements being heated and cooled, including the heat transfer module the thermoelectric module and the heat sink is in the range of 400 grams. Therefore, it takes the 40 watts of cooling capability of the thermoelectric device a long time to cool that entire thermal mass from a high temperature of 40C F to a low temperature of about 5C F.

The thermal therapy device according to the invention avoids the need for the entire cooling system to switch temperatures by maintaining a reservoir of chilled fluid at a low temperature during the entire operating cycle of the unit. Thus, while a resistance heater is heating the tissue being treated the thermoelectric cooling device is energized to maintain a reservoir of chilled fluid at a low temperature of about 40 F. Therefore, when the cooling mode of the therapy is called for, simultaneously the electric resistance heater is turned off and a supply of chilled therapy providing fluid is pumped through the fluid circulation system and reaches the treatment area instantly. Not only is the fluid in the reservoir chilled to the low temperature needed for the cold treatment the reservoir itself has a relatively large thermal mass kept at the low temperature of 40 F, and due to its extended surface area, will readily absorb heat from circulating fluid as it returns from the tissue interacting device, having absorbed heat from the tissue being treated.

An additional advantage to the system according to the invention over the state of the art as exemplified in U.S. '109 lies in the fact that the thermoelectric devices must be driven by low-voltage DC current. It is a known fact that converting AC line-voltage to 12-volt DC requires specialized electronic circuitry known in the trade as “Low Voltage Power Supply” which is relatively expensive, and where the costs increase proportionally to the power rating of the “power supply”. Since the thermoelectric module of the design according to the present invention is used during the entire treatment period: to cool down the cooling block during the heating cycle, and to cool extract heat from the tissue being treated during the cooling cycle its average energy draw is significantly lower than the thermoelectric module in a system according to the U.S. '109, where it must generate the same amount of cooling capacity in about half the time. consequently, the system according to the invention would require less power allowing the use of a less expensive “power supply”.

Additionally, it was determined that monitoring and controlling the temperature of the treated area is of great value as it can forestall injuries caused by over heating or by inflicting “cold-burns” to the skin. It was also determined that the user finds it helpful to control and monitor the treatment area based on their own comfort level. Therefore, the placing of the thermal sensor in the treatment interacting device according to the invention was found to be advantageous.

Although the thermal therapy device has been shown and described with respect to a certain embodiment, it is obvious that equivalent alterations and modifications will occur to others skilled in the art upon the reading and understanding of this specification and the annexed drawings. Regarding the various functions performed by the above described elements (e.g., components, assemblies, systems, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function. In addition, while a feature may have been described above with respect to only one or more of several illustrated embodiments, such a feature may be combined with one or more other features of the other embodiments, as may be desired and advantageous for any given or application.

We claim:

1. A thermal therapy device comprising a tissue interaction portion and a fluid manipulation portion in fluid communication therewith by conduit; wherein
the tissue interacting portion, fluid manipulating portion, and conduit form a closed loop containing a fluid and are adapted to cause the fluid to flow through the closed loop;
the fluid is heated by a heating source and cooled by a cooling source, the heating and cooling sources being distinct from each other;
the tissue interaction portion is adapted to heat and cool patient tissue and includes a temperature sensing device to monitor the temperature of a treatment area and control the heating and cooling sources according thereto.

2. The thermal therapy device according to claim 1 wherein the fluid manipulating portion comprises the heating source, the cooling source, and a pump for causing the fluid to flow through the closed loop.

3. The thermal therapy device according to claim 2 wherein the closed loop comprises channels disposed within the tissue interaction portion and adapted for thermal communication between the fluid and the patient tissue.

4. The thermal therapy device according to claim 1 wherein the heating source is a resistive electric heater.

5. The thermal therapy device according to claim 1 wherein the tissue interactive portion comprises a flexible pad.

6. The thermal therapy device according to claim 2 wherein the cooling source is a thermo electric cooler.

7. The thermal therapy device according to claim 3 wherein the tissue interactive portion comprises a flexible pad.

8. The thermal therapy device according to claim 1 wherein the heating source is a resistive electric heater and the cooling source is a thermo electric cooler.

9. The thermal therapy device according to claim 1 wherein the tissue interactive portion comprises a flexible pad.

10. The thermal therapy device according to claim 1 wherein the fluid manipulating portion comprises the cooling source and a pump for causing the fluid to flow through the closed loop, and the tissue interaction portion comprises the heating source.

11. A thermal therapy device according to claim 10 wherein the conduit includes tubes for passage of tissue treatment fluid and electrical conductors for energizing the heating source.

12. The thermal therapy device according to claim 10 wherein the closed loop comprises channels disposed within the tissue interaction portion and adapted for thermal communication between the fluid and the patient tissue.

13. The thermal therapy device according to claim 11 wherein the heating source is a resistive electric heater.

14. The thermal therapy device according to claim 12 wherein the tissue interactive portion comprises a flexible pad.

15. The thermal therapy device according to claim 11 wherein the cooling source is a thermo electric cooler.

16. The thermal therapy device according to claim 14 wherein the tissue interactive portion comprises a flexible pad.

17. The thermal therapy device according to claim 11 wherein the heating source is a resistive electric heater and the cooling source is a thermo electric cooler.

18. The thermal therapy device according to claim 16 wherein the tissue interactive portion comprises a flexible pad.

19. A thermal therapy device comprising a tissue interaction portion and a fluid manipulation portion in fluid communication therewith by conduit; wherein
the tissue interacting portion, fluid manipulating portion, and conduit form a closed loop containing a fluid and are adapted to cause the fluid to flow through the closed loop;
the fluid is heated by a heating source and cooled by a cooling source; and
the tissue interaction portion is adapted to heat and cool patient tissue and includes a temperature sensing device.

20. A thermal therapy device according to claim 19, wherein the fluid manipulating portion contains electronic controls for processing input from an operator and from the temperature sensing device to manipulate the fluid.

21. A thermal therapy device according to claim 19, wherein the conduit contains fluid passage tubes and electrical conductors.
